(19) United States
(12) Patent Application Publication
(10) Pub. No.: US 2025/0302848 A1
(43) Pub. Date: Oct. 2, 2025

(54) DRUG-IN-CAPSULE FORMULATIONS OF THROMBOXANE RECEPTOR ANTAGONISTS

(71) Applicant: ATXA Therapeutics, Ltd, Dublin 4 (IE)

(72) Inventors: B. Therese Kinsella, Blackrock (IE); Helen Reid, Greystones (IE)

(21) Appl. No.: 19/093,341

(22) Filed: Mar. 28, 2025

Related U.S. Application Data
(60) Provisional application No. 63/571,049, filed on Mar. 28, 2024.

(51) Int. Cl. A61K 31/64; A61K 9/48

(52) U.S. Cl. CPC A61K 31/64; A61K 9/4833; A61K 9/4866

(57) ABSTRACT

The invention provides formulations of certain substituted biphenyl benzenesulfonyl urea compounds, as thromboxane receptor antagonists, in capsules that provide for oral delivery of those drugs with good bioavailability. In particular, compositions of the invention provide certain substituted biphenyl benzenesulfonyl urea compounds (such as N-(tert-butylcarbamoyl)-5-cyano-2-((4′-(trifluoromethoxy)-[1,1′-biphenyl]-3-yl)oxy)benzenesulfonamide) as an active ingredient in an amorphous solid dispersion (ASD) with copovidone. The ASD with a filler, disintegrant and lubricant make up an intragranular blend that, along with an extragranular disintegrant/lubricant blend are encapsulated, making a drug-in-capsule (DiC) formulation.

### TECHNICAL FIELD

The disclosure relates to pharmaceutical compositions and scalable methods for manufacture of drug formulations in capsules that provide oral delivery and good bioavailability of substituted biphenyl benzenesulfonyl urea compounds as thromboxane receptor antagonists.

### BACKGROUND OF THE INVENTION

The thromboxane receptor, also known as the T Prostanoid receptor (TP), is a protein found in humans named for its preferred ligand, thromboxane A2. The TP receptor is encoded by the TBXA2R gene and is found in α and β isoform subtypes. Its ligand, thromboxane (TX) A2 and the free-radical derived isoprostane 8-iso-prostaglandin (PG) F2α signal through the thromboxane receptor (TP) to regulate multiple processes including platelet aggregation, constriction, and proliferation of vascular and pulmonary smooth muscle.

Those ligands also mediate potent pro-inflammatory, pro-mitogenic, and pro-fibrotic effects within the lungs, heart, and wider cardiovascular system. Imbalances in the levels of TXA2, 8-iso-PGF2α, or the TP have been implicated in a range of cardiopulmonary diseases and aberrant TXA2/TP signaling is thought to contribute to cardiac dysfunction.

Synthetic compounds that bind to, but do not activate, TP and thereby inhibit its activation by activating ligands have been considered as potential therapeutic agents for cardiopulmonary diseases such as pulmonary arterial hypertension (PAH), Idiopathic Pulmonary Fibrosis (iPF), other interstitial lung diseases (ILDs) or forms of heart failure. PAH is a rare disease characterized by structural and functional changes in the pulmonary vasculature resulting in elevated pulmonary vascular resistance (PVR), right ventricular (RV) hypertrophy and, ultimately, RV failure and death. ILDs refers to a large group of chronic lung conditions characterized by inflammation and progressive scarring (fibrosis) of the lung, making it increasingly difficult to breathe and, ultimately, leading to respiratory failure. The most common ILD with an unknown cause is iPF.

### SUMMARY OF THE INVENTION

The invention provides formulations of certain substituted biphenyl benzenesulfonyl urea compounds as thromboxane receptor antagonists in capsules that provide for oral delivery of those drugs with good bioavailability. In particular, compositions of the invention provide certain substituted biphenyl benzenesulfonyl urea compounds (such as N-(tert-butylcarbamoyl)-5-cyano-2-((4′-(trifluoromethoxy)-[1,1′-biphenyl]-3-yl)oxy)benzenesulfonamide, hereinafter “NTP42”)) as an active ingredient in an amorphous solid dispersion (ASD) with copovidone. The ASD with a filler, disintegrant and lubricant make up a blend that is encapsulated to make a drug-in-capsule (DiC) formulation. The DiC formulation is (1) able to efficiently and robustly disperse to release its contents in a controlled manner to maximize absorption of the active ingredient after oral dosing; (2) stable to provide an extended shelf-life for the drug product; (3) able to withstand industrial manufacturing processes; and (4) active, in that other ingredients/excipients do not impede the release of or have any detrimental effects on the active drug.

In certain aspects, the invention provides a pharmaceutical composition that includes: a formulation blend comprising (i) an amorphous solid dispersion (ASD) comprising an active ingredient and copovidone, (ii) a filler, (iii) a disintegrant, and (iv) a lubricant, wherein the composition is contained in a capsule and wherein the active ingredient comprises a compound of formula (I):

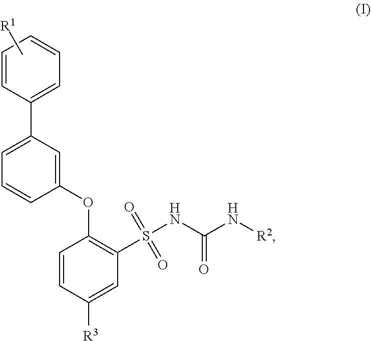

wherein
R¹ is methoxy, R² tert-butyl, and R³ is a nitro group (hereinafter TP20);
R¹ is di-fluoromethoxy, R² tert-butyl, and R³ is a nitrile group (NTP42);
R¹ is tri-fluoromethoxy, R² tert-butyl, and R³ is a nitrile group (hereinafter NTP43);
R¹ is difluoromethyl, R² tert-butyl, and R³ is a nitrile group (hereinafter NTP44);
R¹ is trifluoromethyl, R² tert-butyl, and R³ is a nitrile group (hereinafter NTP45);
R¹ is —CONH₂, R² tert-butyl, and R³ is a nitrile group (hereinafter NTP47);
R¹ is —CONHCH₃, R² tert-butyl, and R³ is a nitrile group (hereinafter NTP48); or
R¹ is a nitrile group, R² tert-butyl, and R³ is a nitrile group (hereinafter NTP49).

In preferred embodiments, the active ingredient is NTP42:

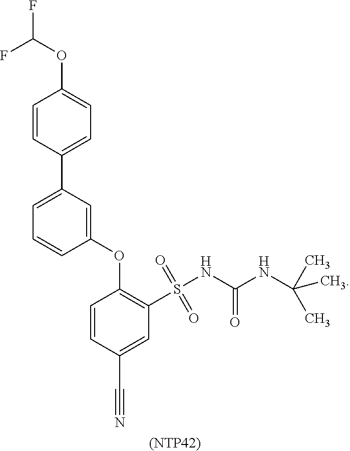 [SMILES: "(NTP42)" ; CC(C)(C)NC(=O)NS(=O)(=O)c1cc(C#N)ccc1Oc1cccc(-c2ccc(OC(F)F)cc2)c1]

The active ingredient and the copovidone may be present in the ASD at a ratio of 1:4 w/w. Preferably, the composition includes: 93.0% the ASD and the filler; 6.0% of the disintegrant; and 1.0% of the lubricant. The amount of ASD may be varied to adjust a dose (e.g., to between about 0.1 and about 90%), with the filler providing the balance of the 93.0%. The excipients may be any of those listed in Table 6; for example, the filler may be mannitol, the disintegrant may be crospovidone, and the lubricant may be magnesium stearate.

Preferably in any or all of the disclosed embodiments the active ingredient is amorphous and the composition does not include any crystal of the active ingredient.

In related aspects, the invention provides a use of any of the active ingredients in making a medicament for treating any of the conditions referred to herein. The medicament is any one of the compositions described herein. The active ingredient may be, for example, one of TP20, NTP42, NTP43, NTP44, NTP45, NTP47, NTP48, or NTP49. The active ingredient is preferably NTP42. In a preferred embodiment, the condition is pulmonary arterial hypertension (PAH).

In certain aspects, the invention provides a method of formulating an active ingredient. The method includes making an amorphous solid dispersion (ASD) comprising the active ingredient and copovidone; forming an blend comprising the ASD, a filler, a first quantity of a disintegrant, and a first quantity of a lubricant; granulating the blend to form a granulated intragranular blend; preparing a mix comprising the intragranular blend and an extragranular blend that comprises a second quantity of the disintegrant and a second quantity of the lubricant; and encapsulating the mix in capsules, wherein the active ingredient comprises one of those specified herein, such as TP20, NTP42, NTP43, NTP44, NTP45, NTP47, NTP48, or NTP49, e.g., preferably NTP42:

In the method, the active ingredient and the copovidone may constitute the ASD at a ratio of 1:4 w/w. In specific embodiments of the method, the mix consists essentially of: the ASD and filler in amounts so that the ASD and the filler are 93.0% w/w of the mix; 3.0% the first quantity of the disintegrant; 0.5% the first quantity of the lubricant; 3.0% the second quantity of the disintegrant; and 0.5% the second quantity of the lubricant. Preferably, in the specific embodiments, the filler is mannitol, the disintegrant is crospovidone, and the lubricant is magnesium stearate. The ASD may be made by making a spray-dried dispersion. The granulating step may be performed via roller compactor.

Aspects of the invention provide a pharmaceutical composition that includes: an intragranular blend comprising (i) an amorphous solid dispersion (ASD) comprising an active ingredient and copovidone, (ii) a filler, (iii) a first quantity of a disintegrant, and (iv) a first quantity of a lubricant; and an extragranular blend comprising (i) a second quantity of the disintegrant and (ii) a second quantity of the lubricant, wherein the composition is contained in a capsule and wherein the active ingredient comprises TP20, NTP42, NTP43, NTP44, NTP45, NTP47, NTP48, or NTP49. The composition may include one or more other excipients. In preferred embodiments, the active ingredient is NTP42. The active ingredient and the copovidone may be present in the ASD at a ratio of 1:4 w/w. Preferably, the composition includes: 93.0% the ASD and the filler; 3.0% the first quantity of the disintegrant; 0.5% the first quantity of the lubricant; 3.0% the second quantity of the disintegrant; and 0.5% the second quantity of the lubricant. The ASD may provide an amount between about 1 and about 90% w/w of the composition, with the filler providing the balance of the 93.0%. In the certain preferred embodiments, the filler is mannitol, the disintegrant is crospovidone, and the lubricant is magnesium stearate. In the certain preferred embodiments, the capsule may be made of hydroxypropyl methylcellulose (HPMC). The compositions may consist essentially of the capsule and contents contained within the capsule. Preferably in any or all of the disclosed embodiments the active ingredient is amorphous.

### BRIEF DESCRIPTION OF DRAWINGS

FIG. 1 gives Table 1, which gives ingredients and quantities.

FIG. 2 gives dissolution profiles.

FIG. 3 illustrates the dissolution profile for Clinical Batches.

FIG. 4 gives Table 2, data from multiple batches.

FIG. 5 gives Table 3 with stability data.

FIG. 6 gives Table 4, showing stability data.

FIG. 7 gives an outline of a clinical trial design.

FIG. 8 gives pharmacokinetic (PK) data.

FIG. 9 gives pharmacodynamic (PD) data.

FIG. 10 gives Table 5, showing excipients tested.

FIG. 11 gives Table 6, showing composition of prototype blends.

FIG. 12 gives Table 7, showing composition of a first round of prototypes.

FIG. 13 gives Table 8, showing reference Carr's Index Values.

FIG. 14 gives Table 9, the Bulk Density and Flow Testing.

FIG. 15 gives a summary of dissolution of certain formulations.

FIG. 16 gives Table 10, showing results from testing.

FIG. 17 gives Table 11, showing bulk density and flow properties.

FIG. 18 gives Table 12, showing data for the Tensile Strength.

FIG. 19 shows dissolution profiles.

FIG. 20 gives Table 13, showing compositions of optimized prototypes.

### DETAILED DESCRIPTION OF THE INVENTION

The disclosure provides formulations for oral, drug-in-capsule delivery of certain sulfonylurea compounds as active ingredients that act as antagonists of the TPα and/or TPβ isoforms of the human thromboxane (TX) A₂ receptor, also referred to as the T prostanoid receptor or, simply as, the TP. The active ingredient of a formulation of the disclosure may be any of the sulfonylurea compounds shown in WO 2022/008515 A1; EP 4178547 A1; US 2023/301946 A1; WO 2016/203314 A1; U.S. Pat. No. 9,932,304 B2; EP 3,310,756 A1; WO 2013/156871 A2; EP2,838,520 A2; or U.S. Pat. No. 9,388,127 B2, the contents of each of which are incorporated by reference, for all purposes. In preferred embodiments, the active ingredient is a compound of formula (I):

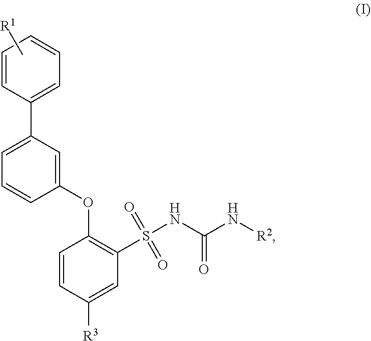

wherein R¹ is a cycloalkyl group, an alkyl group, a heterocycloalkyl group, a difluoromethyl group, a trifluoromethyl group, a halogenated cycloalkyl group, a halogenated alkyl group, a halogenated heterocycloalkyl group, a methoxy group, a halogenated methoxy group, an ethoxy group, an isopropoxy group, a tert-butoxy group, a halogenated ethoxy group, a halogenated isopropoxy group, a halogenated tert-butoxy group, a primary amide (—CONH₂), a secondary amide (—CONHCH₃), a tertiary amide (—CONH(CH₃)₂), or a nitrile group; R² is a linear or branched alkyl group of 2 to 6 carbons or a halogenated alkyl group of 2 to 6 carbons; and R³ is a nitrile group or nitro group.

The active ingredient may preferably one of TP20, NTP42, NTP43, NTP44, NTP45, NTP47, NTP48, & NTP49:

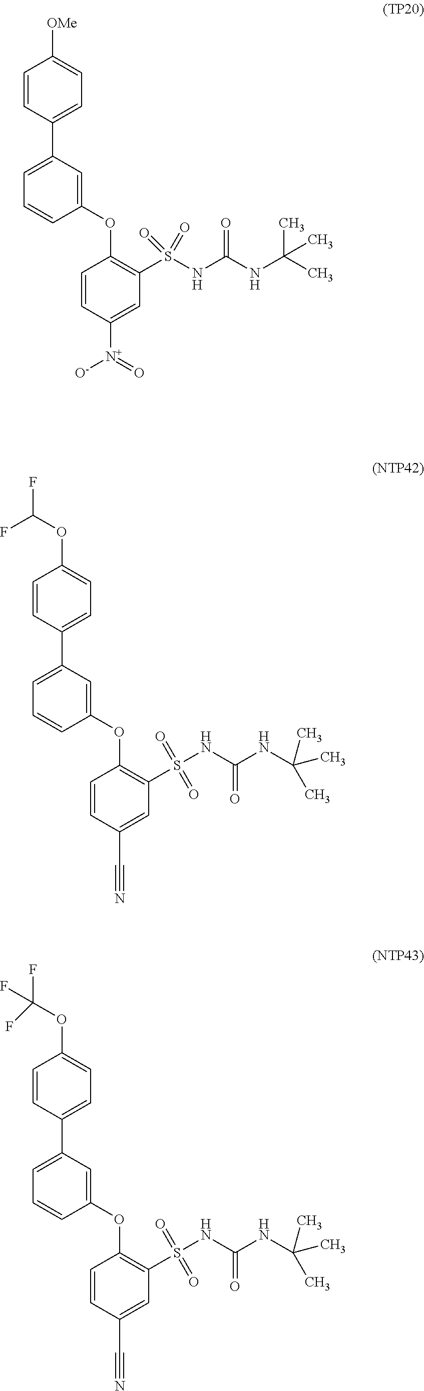 [SMILES: COc1ccc(-c2cccc(Oc3ccc([N+](=O)[O-])cc3S(=O)(=O)NC(=O)NC(C)(C)C)c2)cc1 ; CC(C)(C)NC(=O)NS(=O)(=O)c1cc(C#N)ccc1Oc1cccc(-c2ccc(OC(F)F)cc2)c1 ; CC(C)(C)NC(=O)NS(=O)(=O)c1cc(C#N)ccc1Oc1cccc(-c2ccc(OC(F)(F)F)cc2)c1]

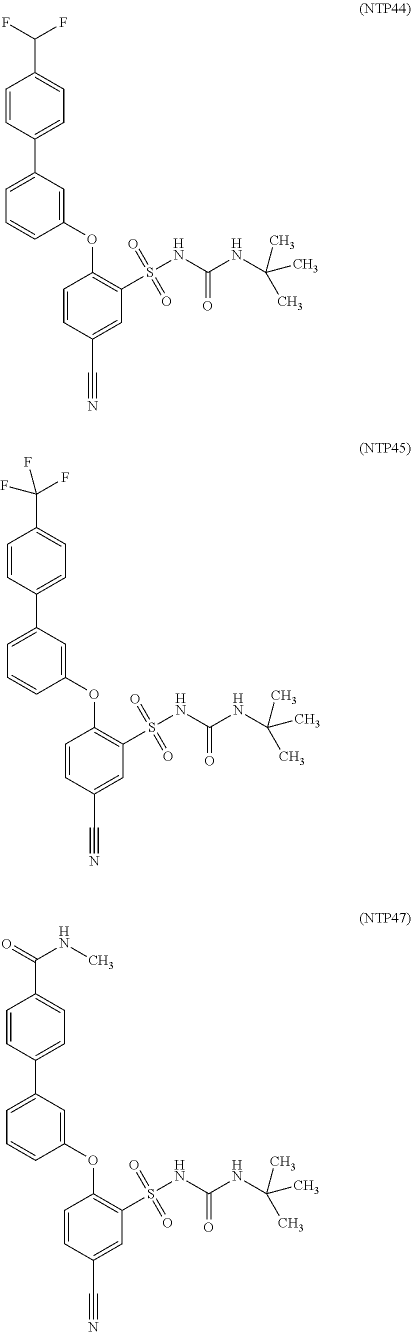 [SMILES: CC(C)(C)NC(=O)NS(=O)(=O)c1cc(C#N)ccc1Oc1cccc(-c2ccc(C(F)F)cc2)c1 ; CC(C)(C)NC(=O)NS(=O)(=O)c1cc(C#N)ccc1Oc1cccc(-c2ccc(C(F)(F)F)cc2)c1 ; CNC(=O)c1ccc(-c2cccc(Oc3ccc(C#N)cc3S(=O)(=O)NC(=O)NC(C)(C)C)c2)cc1]

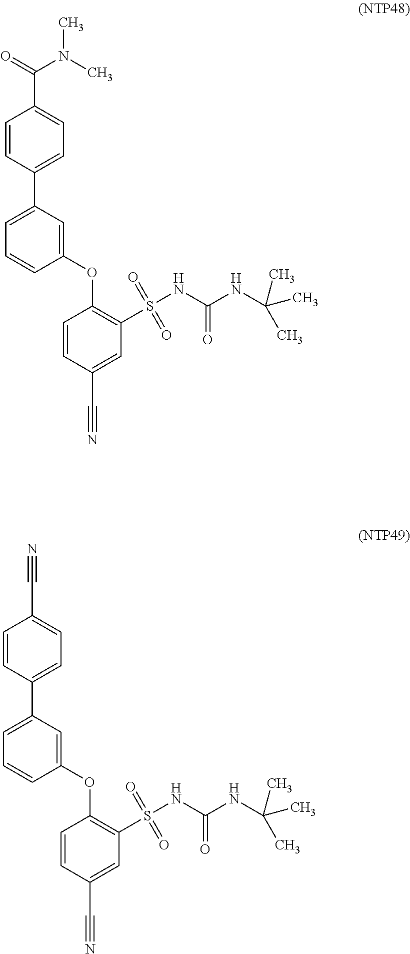 [SMILES: CN(C)C(=O)c1ccc(-c2cccc(Oc3ccc(C#N)cc3S(=O)(=O)NC(=O)NC(C)(C)C)c2)cc1 ; CC(C)(C)NC(=O)NS(=O)(=O)c1cc(C#N)ccc1Oc1cccc(-c2ccc(C#N)cc2)c1]

In certain preferred embodiments, the active ingredient is one of TP20, NTP42, NTP43, and NTP48, which display potent TP antagonist activity, e.g., inhibit aggregation of human platelets ex vivo with IC₅₀ of 1-10 nM; have excellent specificity, pharmacokinetic (PK) & pharmacodynamic (PD) and toxicology profiles; and display efficacy in a range of in vitro, ex-vivo and in vivo pre-clinical models, including in preclinical models of Pulmonary Arterial Hypertension (PAH). In a most preferred embodiment, the active ingredient is NTP42, which is N-(tert-butylcarbamoyl)-5-cyano-2-((4′-(trifluoromethoxy)-[1,1′-biphenyl]-3-yl)oxy)benzenesulfonamide, is in an amorphous solid dispersion (ASD). Clinical trial results show rapid absorption with no adverse events for oral administration of NTP42 as an active ingredient in an ASD with a suitable excipient such as copovidone. The active ingredient may be delivered as 1:4 w/w NTP42/copovidone, such as the copovidone sold on the trademark KOLLIDON® VA 64. Results of such trials are discussed in Reid et al., 2023, Evaluation of NTP42, a novel thromboxane receptor antagonist, in a first-in-human phase I clinical trial, Front Pharmacol 14:1296188, the contents of which are incorporated by reference.

The disclosure describes development of formulation processes to generate and manufacture, at scale, a drug-in-capsule (DiC) drug product with the active ingredient suitable for use via oral administration in humans.

For the target disease indications, e.g., PAH, other cardiopulmonary diseases and heart failure, oral delivery may be a preferred route of administration of an active ingredient such as NTP42. A suitable formulation includes an active ingredient such as NTP42 spray dried with the pharmaceutically acceptable vinylpyrrolidone-vinyl acetate copolymer KOLLIDON® VA 64 to produce a high-quality drug product, e.g., a Spray Dried Dispersion (SDD) referred to as NTP42:KVA4 where KVA is an abbreviation for KOLLIDON® VA 64 and, for this particular formulation, the variable number 4 specifies that the ratio of NTP42: KOLLIDON VA 64 is 1:4 (w/w). The Spray Dried Dispersion (SDD)-based formulation is used in scaled-up manufacture and testing in a trial where it has been given to healthy male volunteers as an oral suspension (drug-in-suspension or DiS). NTP42:KVA4, when given orally as single (0.25 mg-243 mg NTP42:KVA4) or repeated (15 mg-135 mg NTP42:KVA4 once daily for 7 days) doses was deemed safe and well-tolerated with excellent drug exposure and with a half-life of ˜ 18 h. PK analyses confirmed good drug uptake after oral dosing, where plasma levels of NTP42 increased in a dose-dependent manner, with no food affect and with no drug accumulation observed after repeated dosing. Moreover, as a surrogate marker for efficacy and evidence of drug-target engagement, platelet aggregation assays confirmed PD effects, whereby plasma levels of NTP42 led to inhibition of aggregation induced by the thromboxane agonist U46619, but not that of the purinergic receptor agonist, ADP (adenosine diphosphate). Results indicate excellent correlation between the PK and PD effects, with the time taken for U46619-induced platelet aggregation to return to pre-dose or baseline levels directly correlating to the NTP42 plasma levels. The invention now provides an optimized formulated drug product, with NTP42:KVA4 as a suitable dose form or ‘drug-in-capsule’ (DiC) for oral dosing. Aspects of the invention provide methods of making a drug-in-capsule formulation of an active ingredient such as one of those shown herein.

The invention provides a method of making a drug-in-capsule formulation of an active ingredient such as one of those shown herein, e.g., an ASD of NTP42:KVA4. Preferably all steps, discussed in greater detail below, are performed under GMP conditions and are adaptable for adjustments in dose strength, such as 10 mg, 30 mg or 120 mg NTP42:KVA4 capsules.

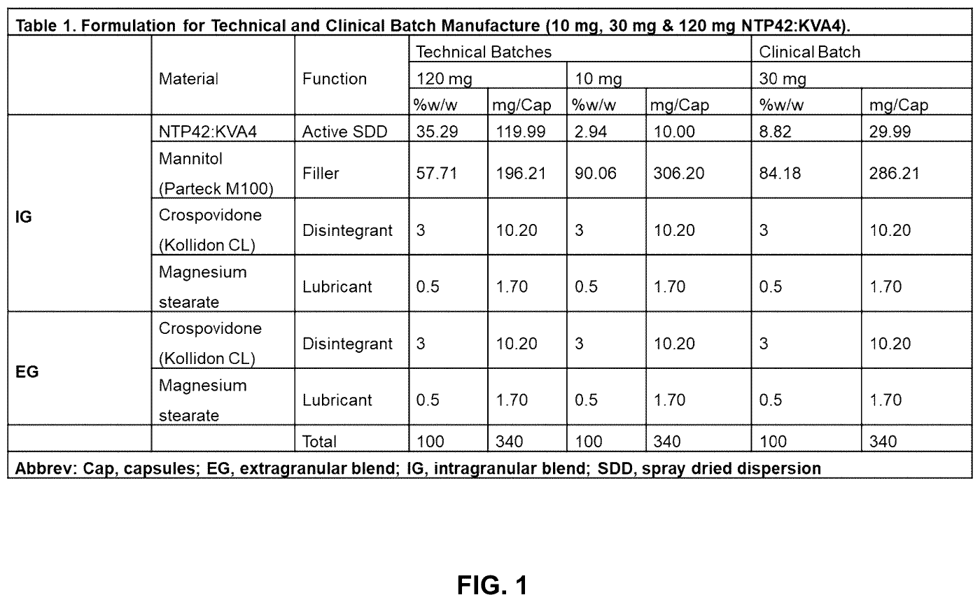

FIG. 1 shows Table 1, which gives ingredients and quantities for certain exemplary technical and clinical batches that will be discussed, described, and referred to. As used herein, together all of the active ingredients and all of the excipients that go into the capsule are referred to as the mix. Within the mix, the active ingredient, or drug, is present in an active spray dried dispersion (SDD), aka an amorphous solid dispersion (ASD). Any suitable active ingredient shown herein may be used. In preferred embodiments, the active ingredient is NTP42:

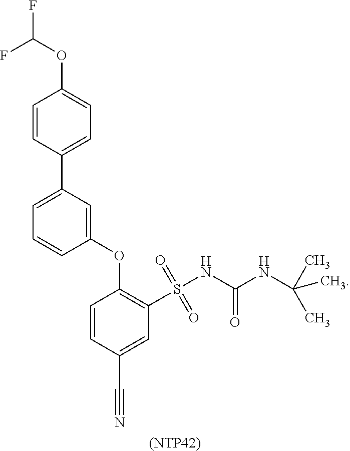 [SMILES: "(NTP42)" ; CC(C)(C)NC(=O)NS(=O)(=O)c1cc(C#N)ccc1Oc1cccc(-c2ccc(OC(F)F)cc2)c1]

Preferably, the active ingredient and the copovidone may be present in the ASD at a ratio of 1:4 w/w. In certain embodiments, the composition includes: 83.0%-93.0% the ASD and the filler; 6.0%-9.0% of the disintegrant; 1.0% of the lubricant. The amount of ASD may be varied to adjust dose, with the filler providing the balance of the 83.0%-93.0% ASD and filler. The filler may be mannitol, lactose monohydrate, microcrystalline cellulose; the disintegrant may be sodium starch glycolate, crospovidone; and the lubricant may be magnesium stearate.

In certain embodiments, the invention provides a pharmaceutical composition that includes: a blend comprising (i) an amorphous solid dispersion (ASD) comprising an active ingredient and copovidone, (ii) a filler, (iii) a disintegrant, (vi) a poloxamer agent, and (v) a lubricant, wherein the composition is contained in a capsule. The filler may be mannitol, lactose monohydrate, microcrystalline cellulose; the poloxamer agent may be poloxamer 188; the disintegrant may be sodium starch glycolate, crospovidone; and the lubricant may be magnesium stearate.

In certain embodiments, the invention provides a pharmaceutical composition that includes: a blend comprising (i) an amorphous solid dispersion (ASD) comprising an active ingredient and copovidone, (ii) a filler, (iii) a disintegrant, (vi) an anti-gelling agent, and (v) a lubricant, wherein the composition is contained in a capsule. The filler may be mannitol, lactose monohydrate, microcrystalline cellulose; the anti-gelling agent may be sodium chloride; the disintegrant may be sodium starch glycolate, crospovidone; and the lubricant may be magnesium stearate.

In certain embodiments, the invention provides a pharmaceutical composition that includes: a blend comprising (i) an amorphous solid dispersion (ASD) comprising an active ingredient and copovidone, (ii) a filler, (iii) a disintegrant, (vi) an anti-gelling agent, (v) a surfactant, and (vi) a lubricant, wherein the composition is contained in a capsule. The filler may be mannitol, lactose monohydrate, microcrystalline cellulose; the anti-gelling agent may be sodium chloride; the surfactant may be sodium laurate sulfate; the disintegrant may be sodium starch glycolate, crospovidone; and the lubricant may be magnesium stearate.

In certain aspects, the invention provides a pharmaceutical composition that includes: a blend comprising (i) an amorphous solid dispersion (ASD) comprising an active ingredient and copovidone, (ii) a filler, (iii) a disintegrant, (iv) a glidant, and (vi) a lubricant, wherein the composition is contained in a capsule. The filler may be mannitol, lactose monohydrate, microcrystalline cellulose; the disintegrant may be sodium starch glycolate, crospovidone; the glidant may be silicon dioxide; and the lubricant may be magnesium stearate.

In preferred embodiments, the ASD is preferably 1:4 (w/w) NTP42:copovidone. The mix may consist essentially of the ASD, where the amount of ASD may vary to adjust the dose and with the filler in an amount so that the ASD and the filler are 93.0% w/w of the mix; 6.0% of the disintegrant; 1.0% of the lubricant. Preferably, in the specific embodiments, the filler is mannitol, the disintegrant is crospovidone, and the lubricant is magnesium stearate. Mannitol may be preferred as a filler due its non-hygroscopicity, rapid disintegration and high dilution properties. Moreover, the Partek® M100 Mannitol is a direct compression grade of mannitol that may be preferred due to its ability to withstand compression forces and its suitability for wet and dry granulation approaches, with dry granulation being preferred or the method of preparing the present DiC NTP42:KVA4. Crospovidone may be used as a disintegrant, absorbing water and swelling to break apart solid drug forms, such as after oral dosing, facilitating dissolution of the drug from tablets or capsules. Magnesium stearate may be used as an anti-adherent or lubricating agent.

As stated, the formulation blend used in the DiC NTP42:KVA4 provides an immediate release form of the active ingredient or drug product. Low to high dose strength capsules may be manufactured. By way of example, during development, low and high dose strengths, namely at 10 mg & 120 mg NTP42:KVA4, equivalent to 2 mg and 24 mg NTP42, respectively, were manufactured (Table 1). Flexibility in the range of dose strengths is possible for the DiC NTP42:KVA4. For example, batches of the DiC drug product at 10 mg (2 mg NTP42), 30 mg (6 mg NTP42) and 120 mg (24 mg NTP42) NTP42:KVA4 have been manufactured and tested. Technical and Clinical Batches of the DiC NTP42:KVA4 have been blended at 1 kg and 2.4 kg scale, respectively, following the batch formula defined in Table 1.

The method includes making or obtaining a formulation blend with the amorphous solid dispersion (ASD) comprising the active ingredient and copovidone and other ingredients/excipients. The required amounts of ASD and excipient materials are dispensed and the method includes forming an intragranular blend comprising the ASD, a filler, a first quantity of a disintegrant, and a first quantity of a lubricant. In the method, the SDD NTP42:KVA4 is blended with pharmaceutical grade excipients, including a mannitol-based filler (i.e., Partek® M100 Mannitol) and a crospovidone disintegrant (i.e., Kollidon® CL) to produce an intragranular blend. Blend components are layered (e.g., 33% Filler, 50% NTP42:KVA4, 50% Partek® M100 Mannitol, 50% other excipients, 33% Filler, 50% NTP42:KVA4, 50% Partek® M100 Mannitol, 50% other excipients and 33% Filler) and blended for 15 min at 30 rpm using a blender (e.g., Erweka blender). Blend uniformity is evaluated before progressing to the next step.

The intragranular blend is then sieved and a lubricant (i.e., magnesium stearate, Ligamed® MF-2-V) is added before roller compaction using a roller compactor (e.g., Fitzpatrick CCS320 roller compactor) to provide granules. The formulation blend, or mix, for the intragranular blend of a spray-dried dispersion (SDD) or ASD of NTP42:KVA4 with the excipients filler such as mannitol (Partek® M100), disintegrant such as crospovidone (KOLLIDON® CL) and lubricant such as magnesium stearate need not have additional excipients present. The method includes granulating the blend, e.g., via roller compaction of the intragranular material, to form a granulated intragranular blend.

Using a dry granulation approach, the NTP42:KVA4/excipient blend is granulated using the roller compactor. Following dry granulation of the SDD with the intragranular excipients, an extragranular (EG) disintegrant such as crospovidone (Kollidon® CL) and EG lubricant such as magnesium stearate are screened, measured, and blended to prepare the mix comprising the intragranular blend and an extragranular blend that comprises a second quantity of the disintegrant and a second quantity of the lubricant.

In certain embodiments, the extragranular Kollidon® Cl is blended with the granules prior to the addition of the extragranular magnesium stearate and final blending. A required number of capsules and the mix are dispensed and the method includes encapsulating the mix in capsules using a suitable instrument such as an encapsulation machine (e.g., Bonapace IN-CAP). Blend uniformity is confirmed before the granulated blend is encapsulated using automated capsule filling into capsules (e.g., Size 0, Swedish Orange, HPMC capsules; target capsule fill weight 340±35.5 mg). In certain embodiments, NTP42:KVA4 capsules are filled into 60 mL high density polyethylene (HDPE) bottles (e.g., DUMA®) and sealed with a PP cap containing integral desiccant. The blending and granulation processes have been optimized for scaled-up manufacture, where multiple batches of high-quality NTP42:KVA4 capsules have been successfully manufactured.

The method provides a solid oral dose form of NTP42:KVA4 suitable for use by patients outside of the clinical setting. From several prototypes, a drug-in-capsule (DiC) form of NTP42:KVA4 was selected to bring forward for scaled-up manufacture and evaluation in a Bridging clinical trial in healthy volunteers to validate it as suitable for use in a Phase II clinical trial in PAH patients.

The drug-in-capsule (DiC) formulations of the active ingredient (e.g., any one of the thromboxane receptor antagonists shown herein) are provided for their favorable dissolution profiles and good bioavailability. Without being bound by any mechanism, the substituted biphenyl benzenesulfonyl urea compounds shown herein may be prone to crystallization in a manner that inhibits bioavailability and bioavailability may be optimized by keeping those compounds in an amorphous (i.e., not crystalline) form, which may be best achieved by the avoidance or exclusion of water. DiC formulations of the disclosure should provide good dissolution profiles compatible with good bioavailability.

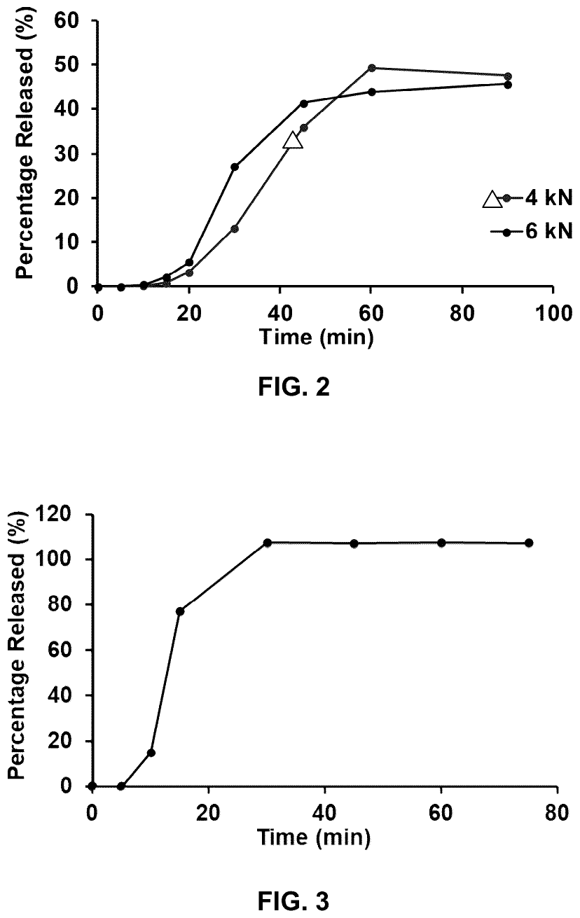

FIG. 2 gives dissolution profiles of 120 mg NTP42:KVA4 Capsules (Prototype #1; Comparison of Roll Force). As illustrated, the DiC form of NTP42:KVA4 is shown to efficiently release NTP42 from the capsules with a favorable dissolution profile and no evidence of precipitation/re-crystallization in the biorelevant media, e.g., Fasted State Simulated Intestinal Fluid (FaSSIF; pH 6.5). Dissolution in phosphate buffer is used to test the ability of the capsules to disperse NTP42 during formal release and stability testing.

FIG. 3 illustrates the dissolution profile for Clinical Batch release testing for the 30 mg NTP42:KVA4 capsules. For formal release of the capsulated drug product, the acceptance criterion for dissolution is specified as Q (quantity of NTP42 released)=80% at 45 minutes. As demonstrated, the NTP42:KVA4 capsules disperse NTP42 rapidly with the quantity released >100% at 30 min, more than meeting the acceptance criteria.

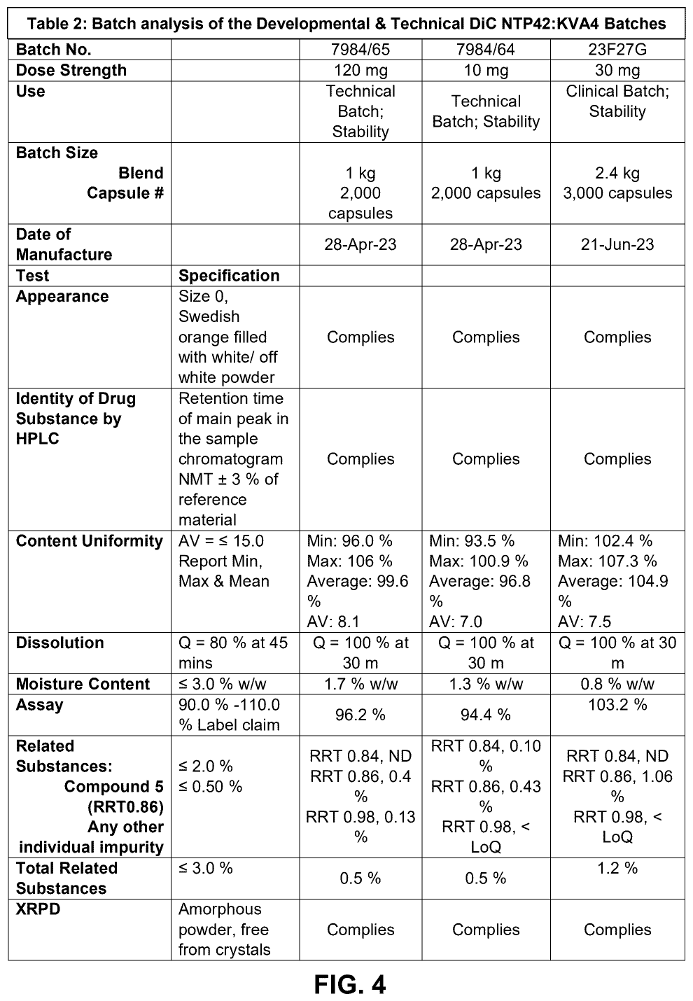

FIG. 4 gives Table 2, showing analytical data from multiple batches at different dose strengths that have been manufactured.

On completion of release testing samples of the Technical and Clinical Batches of the DiC NTP42:KVA4 were set-down for long-term stability under refrigerated (2-8° C.), 25° C./60% relative humidity (RH) and 40° C./75% RH International Council for Harmonisation (ICH) storage conditions. Available data confirm that the capsules are stable with a shelf-life of at least 12 months from date of manufacture based on 3 months set-down refrigerated and 25° C./60% RH storage conditions.

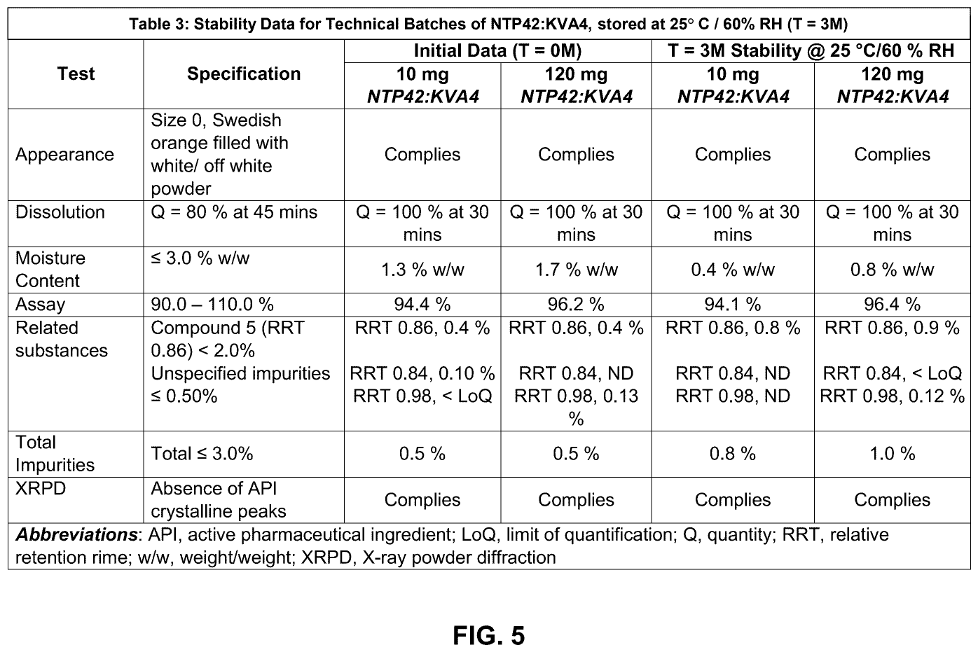

FIG. 5 presents Table 3, stability data for technical batches of DiC NTP42:KVA4 stored at 25° C./60% RH for 3 months (T=3M).

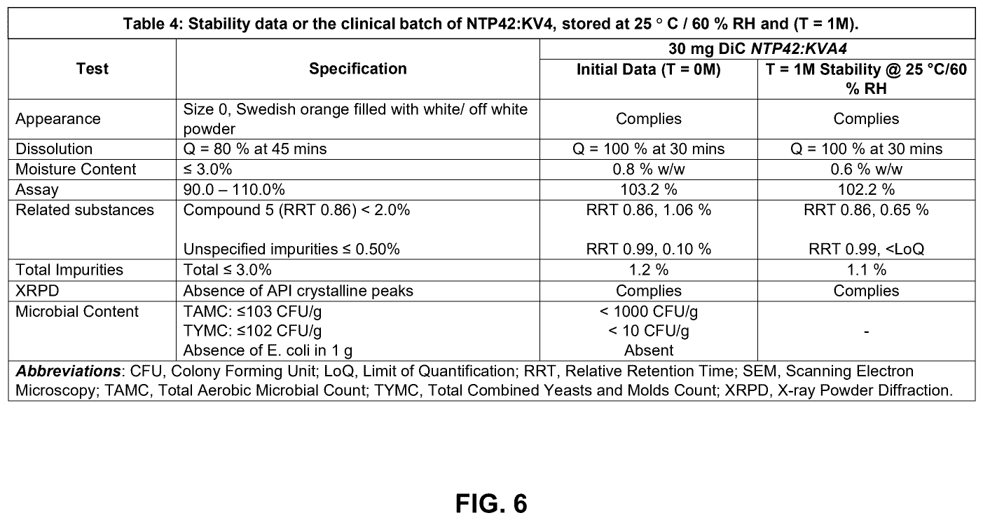

FIG. 6 presents Table 4, stability data for Clinical Batches of DiC NTP42:KVA4 stored at 25° C./60% RH for 1 month (T=1M).

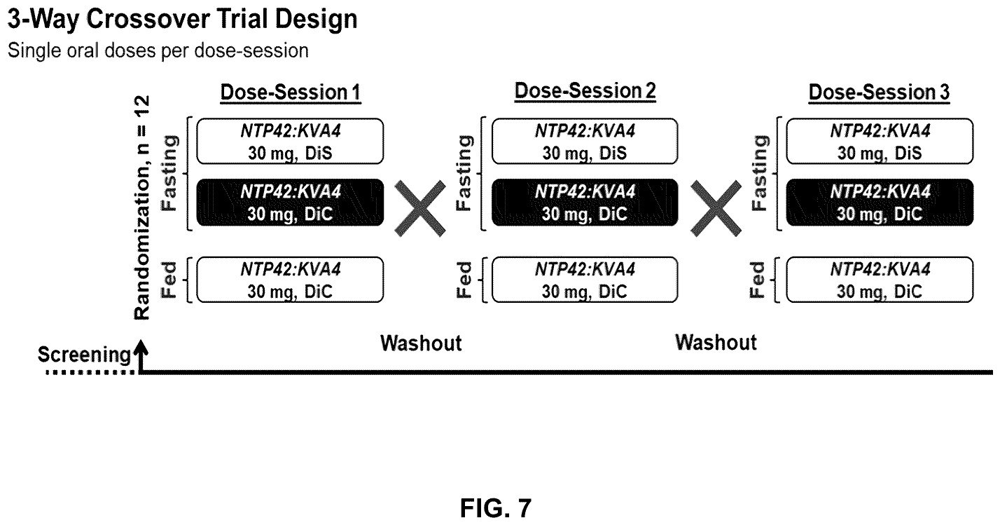

FIG. 7 gives an outline of a clinical trial design for testing the DiC NTP42:KVA4. As outlined, the trial involves a 3-way cross-over design with twelve subjects each receiving three single doses of 30 mg NTP42:KVA4, namely, as an oral suspension (drug-in-suspension or DiS form) to fasted subjects, as the DiC form given to fasted subjects and to subjects after a full breakfast (fed state). On the day of dosing (Day 1) and for all of the three dose sessions, each participant was given NTP42:KVA4, either the DiS or DiC form as per the treatment sequence. The trial confirms that when given orally as either the oral suspension (DiS) or the DiC form, NTP42:KVA4 was deemed safe and well-tolerated.

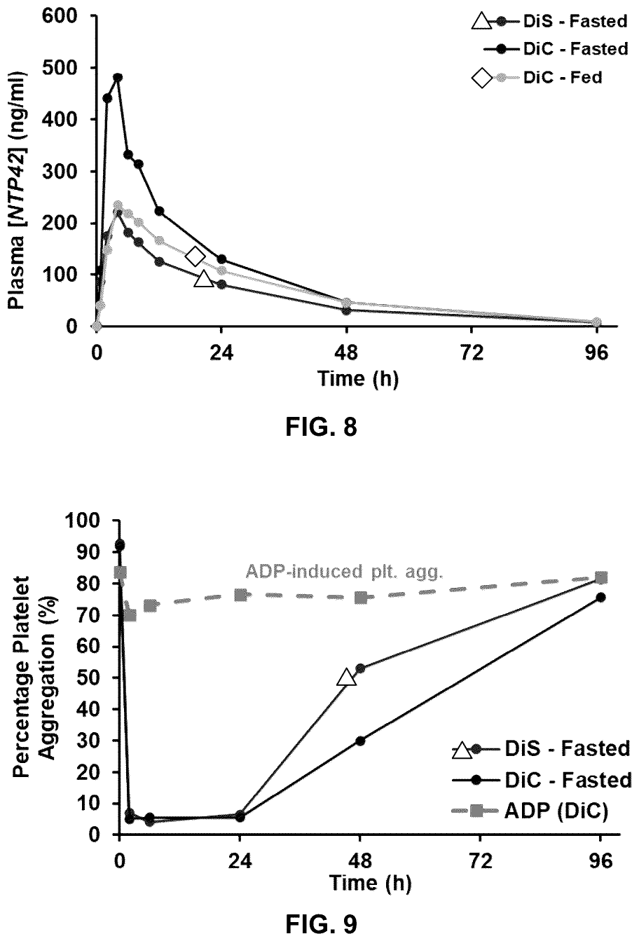

FIG. 8 gives pharmacokinetic (PK) data from the trial. As shown after DiC NTP42:KVA4 dosing, NTP42 is rapidly absorbed with controlled release of the drug from the capsule. Plasma levels of NTP42 after dosing with the oral suspension (DiS) were much more variable and were generally less than that offered by the DiC NTP42:KVA4. The clearance rate of NTP42 from the body was similar after DiS and DiC dosing with the half-life for elimination found to be approx. 15-20 hours. Thus, the DiC NTP42:KVA4 provides excellent drug exposure and with favorable clearance rates that make it suitable for once daily oral dosing.

FIG. 9 gives Pharmacodynamic (PD) Data from the trial. As shown, when given as either the DiS or DiC form, NTP42:KVA4 dosing led to rapid and sustained inhibition of U46619-, but not ADP-induced platelet aggregation, confirming selective target engagement where the drug NTP42 specifically binds and inhibits (antagonizes) its drug target, namely the human thromboxane receptor or TP, without non-selectively affecting the control ADP receptor that is also found on platelet membranes. Moreover, it was also confirmed that some 5 days after each NTP42:KVA4 dose, U46619-induced platelet responses had returned to baseline or pre-dose levels consistent with its effective drug clearance by the body.

Consistent with the disclosed methods, the invention provides pharmaceutical compositions that include an intragranular blend comprising (i) an amorphous solid dispersion (ASD) comprising an active ingredient and copovidone, (ii) a filler, (iii) a first quantity of a disintegrant, and (iv) a first quantity of a lubricant; and an extragranular blend comprising (i) a second quantity of the disintegrant and (ii) a second quantity of the lubricant.

Making reference to formula (I):

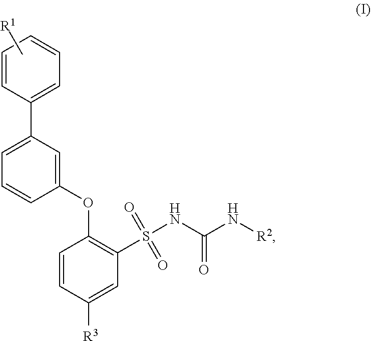

the active ingredient may be one of TP20, NTP42, NTP43, NTP44, NTP45, NTP47, NTP48, & NTP49, where for TP20, R¹ is methoxy, R² tert-butyl, and R³ is a nitro group; for NTP42, R¹ is di-fluoromethoxy, R² tert-butyl, and R³ is a nitrile group; for NTP43, R¹ is tri-fluoromethoxy, R² tert-butyl, and R³ is a nitrile group; for NTP44, R¹ is difluoromethyl, R² tert-butyl, and R³ is a nitrile group; for NTP45, R¹ is tri-fluoromethyl, R² tert-butyl, and R³ is a nitrile group; for NTP47, R¹ is a primary amide (—CONH₂), R² tert-butyl, and R³ is a nitrile group; for NTP48, R¹ is a secondary amide (—CONHCH₃), R² tert-butyl, and R³ is a nitrile group; and for NTP49 R¹ is a nitrile group, R² tert-butyl, and R³ is a nitrile group.

In a preferred embodiment, the active ingredient is NTP42:

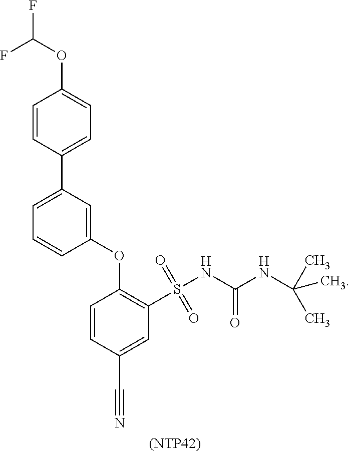 [SMILES: "(NTP42)" ; CC(C)(C)NC(=O)NS(=O)(=O)c1cc(C#N)ccc1Oc1cccc(-c2ccc(OC(F)F)cc2)c1]

In pharmaceutical compositions of the invention, the active ingredient and the copovidone may be present in the ASD at a ratio of 1:4 w/w. Certain embodiments include TP antagonists (e.g., TP20, NTP42, NTP43 & NTP48) that inhibit the actions of TXA₂ and of the free-radical derived isoprostane 8-iso-prostaglandin (PG) F_{2α}, in addition to certain other incidental ligands (e.g., the endoperoxide PGG₂/PGH₂) each of which act as agonists or partial agonists of the TP. The TP is expressed in a range of specific cell types throughout the body and its expression is altered in several disease indications. Active ingredients described herein such as NTP42 target the TPs (including TPα and/or TPβ) expressed in each of those cell types and in different disease settings.

Compositions of the disclosure may be used in the treatment of other diseases in which TXA₂, 8-iso-PGF_{2α} or the TP receptor itself have been implicated. Those conditions include, but are not limited to, various other cardiovascular (thrombosis, various hypertensions including systemic and pregnancy induced hypertension, arterial peripheral disease), pulmonary (including asthma, Chronic obstructive pulmonary disease (COPD), Idiopathic Pulmonary Fibrosis (iPF) or other interstitial lung diseases (ILDs)), heart failure and renal (including glomerular nephritis & renal hypertension) diseases. The compositions also have potential applications in the treatment of prostate disease (such as benign prostate hyperplasia), various pro-inflammatory (including, but not limited, to inflammatory CVD, renal, pulmonary, post-viral/microbial infection) and neoplastic (e.g., breast, lung or prostate cancers including castrate-resistant prostate cancer (CRPC)) diseases.

The invention described herein, i.e., drug-in-capsule (DiC) forms of an ASD comprising a TP antagonist and KVA, e.g., the DiC NTP42:KVA4, provides an easy and convenient solid oral dose form for patients to self-administer outside of the clinical environment and has applications in a wide variety of disease settings. In certain embodiments (see Table 1), the composition includes 93.0% the ASD and the filler; 3.0% the first quantity of the disintegrant; 0.5% the first quantity of the lubricant; 3.0% the second quantity of the disintegrant; and 0.5% the second quantity of the lubricant. In the 93.0% that is the ASD+filler, to allow for different dosages of the active ingredient, the ASD may be varied in the composition with the filler providing the balance of the 93.0%. In certain embodiments, the filler may be mannitol, the disintegrant may be crospovidone, and the lubricant may be magnesium stearate.

In certain preferred embodiments, e.g., as shown in Table 1, the contents of the capsule consist of: 316.20 mg ASD and filler; 10.20 mg for the first quantity of the disintegrant; 1.70 mg for the first quantity of the lubricant; 10.20 mg for the second quantity of the disintegrant; and 1.70 mg for the second quantity of the lubricant, wherein the 316.20 mg ASD and filler comprises an amount of ASD (e.g., 10.00 mg, 29.9 mg or 119.99 mg), with the filler being the balance of the 316.20 mg. Preferably, in the preferred embodiments, the filler is mannitol, the disintegrant is crospovidone, and the lubricant is magnesium stearate. The capsule may be made of hydroxypropyl methylcellulose (HPMC). In preferred embodiments throughout herein, the active ingredient is preferably amorphous and the composition does not include any crystal of the active ingredient.

The skilled artisan will recognize that features relevant to the invention may be further described in Reid et al., 2023, Evaluation of NTP42, a novel thromboxane receptor antagonist, in a first-in-human phase I clinical trial, Front Pharmacol 14:1296188; WO 2022/008515 A1; EP 4178547 A1; US 2023/301946 A1; WO 2016/203314 A1; U.S. Pat. No. 9,932,304 B2; EP 3,310,756 A1; WO 2013/156871 A2; EP2,838,520 A2; or U.S. Pat. No. 9,388,127 B2; Bhujbal, 2021, Pharmaceutical amorphous solid dispersion: a review of manufacturing strategies, Acta Pharma Sincia B 11(8):2505-2536; Buhler, 2008, Kollidon Polyvinylpyrrolidone excipients for the pharmaceutical industry, 9th Rev Ed, BASF, Ludwigshafen, DE (331 pages); Partik, 2019, A brief review on Kollidon, J Drug Deliv Ther 9(2):493-500; Kosugi, 2020, Effect of different direct compaction grades of mannitol on the storage stability of tablet properties investigated using a Kohonen self-organizing map and elastic net regression model, Pharmaceutics 12(9):886, the contents of each of which are incorporated for all purposes.

But for compositions described herein, there is a risk of capsules gelling on wetting in biorelevant media and impeding the efficient release of the SDD powder (aka ASD) followed by the release of NTP42 from the Kollidon® VA 64 polymer. Such capsule gelling appears to occur randomly and would lead to a delay and to variability in the dissolution of the active ingredient without compositions described herein. When dosed orally, that gelling effect would impede the release of the drug and overall absorption in the body, making the drug unavailable. Here, compositions of the disclosure minimize or avoid the gelling effect and promote the efficient, controlled release of the drug from the capsule.

Additionally, HPMC or gelatin capsules are hygroscopic in nature and, without compositions described herein, would not offer protection of the capsule fill from moisture ingress. By way of example, Barham et al., 2015 (Int J Pharmaceutics 478:796-803) compared the moisture permeability of HPMC and gelatin capsules as well as their ability to protect fill contents from moisture ingress. In that study, spray dried amorphous lactose in both HPMC and gelatin capsules re-crystallized when they were exposed to high humidity conditions. Similarly, without formulations of the disclosure, the SDD NTP42:KVA4 may be prone to hydrolysis to an inert precursor as well as re-crystallization. Compositions of the disclosure protect the active ingredient from humidity, avoid crystallization of the active ingredient (e.g., NTP42), and provide a suitable solid oral dose form with a long shelf-life.

It is noted that amorphous solid dispersions (ASDs), including SDDs such as NTP42:KVA4, prepared by spray drying, if those were not provided with excipients such as those described herein, would potentially be unsuitable for large-scale industrial capsule filling processes. The SDD drug product must withstand any mechanical processes needed to increase its density or improve its flowability to ensure accurate and robust capsule filling. Excipients of the disclosure are capable of withstanding mechanical mixing and compaction forces during the preparation of the formulation for capsule filling and provide for the stability of the SDD and for accurate filling of the final drug product.

The specific excipients used in the selected lead DiC formulation were selected from compatibility evaluations (physical & chemical stability) and testing of prototype formulations. From those detailed analyses, prototypes were selected for further optimization to ensure that the DiC form would provide efficient drug release (dissolution) and be stable. Notably, compositions of the disclosure provide DiC formulations that allow for a range of dose strengths using the same excipients and formulation processes.

### EXAMPLES

### Example 1: Formulation Blends

To develop a DiC form of NTP42:KVA4, its compatibility with a range of pharmaceutical excipients routinely used in immediate release capsules, including fillers, glidants, disintegrants, anti-gelling agents, surfactants and lubricants with low moisture content, was assessed.

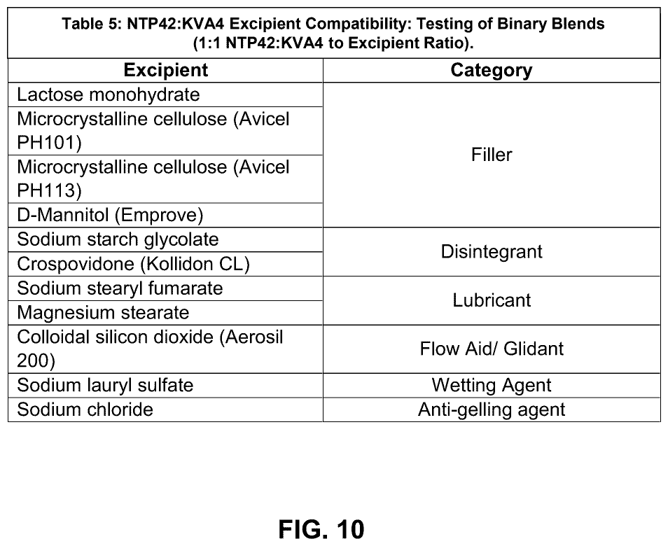

FIG. 10 gives Table 5, showing excipients tested in binary blends (1:1 NTP42:KVA4 to Excipient Ratio). The particle size grades of filler were selected based on suitability for dry granulation with finer grades used to ensure good compaction for a granule formation. Initially, excipient compatibility in binary mixtures (1:1 NTP42:KVA4 to excipient ratio; Table 5) and in prototype type blend formulations were assessed in an excipient compatibility sample matrix.

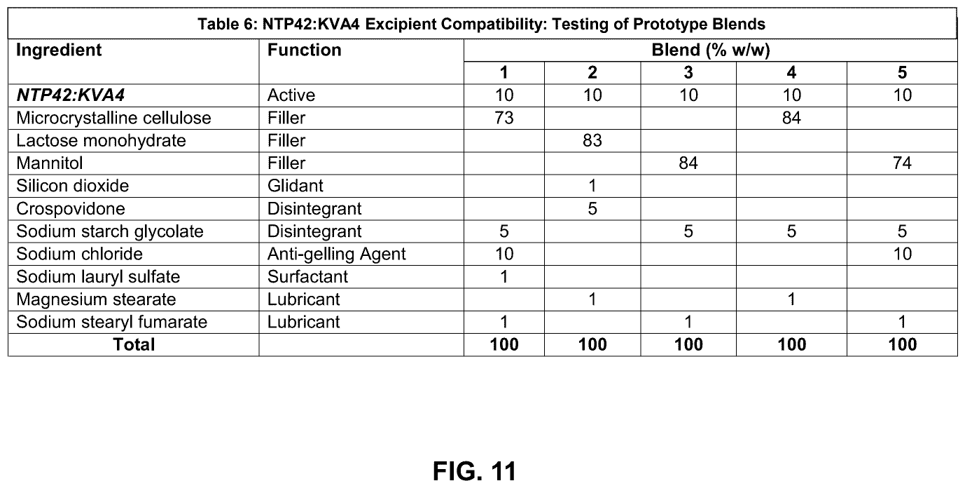

FIG. 11 gives Table 6, showing composition of prototype blends assessed in testing excipient compatibility with NTP42:KVA4. NTP42:KVA4 binary and prototype blend formulations were assessed for stability after 8-weeks storage at refrigerated (2-8° C.) and at 25° C./60% RH conditions.

Due to an impurity observed in binary and multi-component blends containing microcrystalline cellulose (MCC), eluting closely to one inert impurity referred to as Compound 5 but unrelated to NTP42 or NTP42:KVA4, this excipient (MCC) was not progressed for further development. As such, reference to compositions that include other excipients, or even to compositions that “consist essentially of” specified ingredients, can be understood to include the proviso that the described composition contains no microcrystalline cellulose (MCC). As discussed separately below, such compositions could include, e.g., Kolliphor® P188 without deviating from the scope of the disclosure. For the other binary and multi-component blends, while Compound 5 levels increased after 8-weeks stability storage, levels were comparable to the SDD control and, notably any changes in Compound 5 levels were less pronounced in the multi-component blends.

The physical stability of each blend was assessed by X-ray powder diffraction (XRPD) and confirmed that the NTP42:KVA4 in all binary and multi-component blends remained amorphous with no new crystal peaks observed following storage at refrigerated or at 25° C./60% RH conditions. Any peaks present in the XRPD diffractograms of the binary or multi-component blends were attributed to the excipients present and did not change after 8 weeks set down at each of the stability storage conditions. This result is notable. Compositions of the disclosure have been shown to include NTP42 only in amorphous form, with no crystals.

Thus, apart from MCC, NTP42:KVA4 is physically and chemically compatible with the pharmaceutical excipients tested.

Initial formulations, composed of the base components NTP42:KVA4, a filler, a disintegrant and lubricant, were manufactured 4.5 g scale at the highest dose (120 mg NTP42:KVA4) by slugging to produce granules in a “first round” of prototype formulation blends.

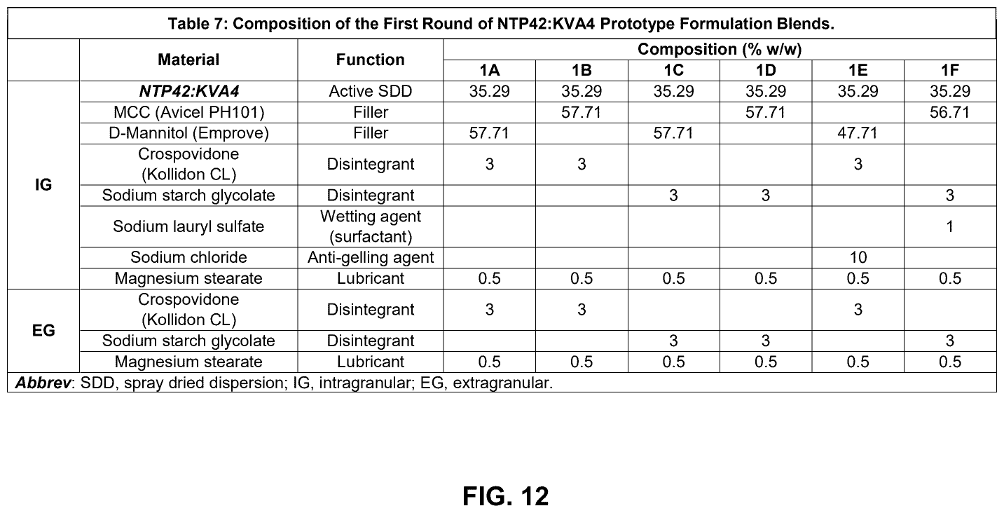

FIG. 12 gives Table 7, showing composition of the First Round of NTP42:KVA4 Prototype Formulation Blends. The ratios of the excipients in each formulation in the first round of screening were decided based on typical levels for formulating immediate release capsules. The prototype formulations were hand-filled into capsules and evaluated for their dissolution profiles in FaSSIF, pH 6.5.

To prepare the formulations, intragranular excipients were layered, before blending and slugging to generate granules. Note that slugging is a dry granulation method where the powder is compacted using a tablet press. The compact is then broken down into granules manually with a pestle and mortar.
⅓ Filler
Half SDD.
Half disintegrant.
Half of other excipients (As applicable).
⅓ Filler.
Half of SDD
Half of disintegrant.
Half of other excipients (As applicable)
⅓ Filler

Prior to hand-filling into the capsules, the formulation blends were assessed for bulk density and determination of the Carr's index, a measure of compressibility and flowability of a powder. Carr's index is calculated based on true and bulk density of the powder using a formula for the Carr Index. The formula for the Carr Index is

$$Carr Index ( \% ) = ( ( Tapped Density - Poured Density ) / ( Tapped Density ) ) \times 100.$$

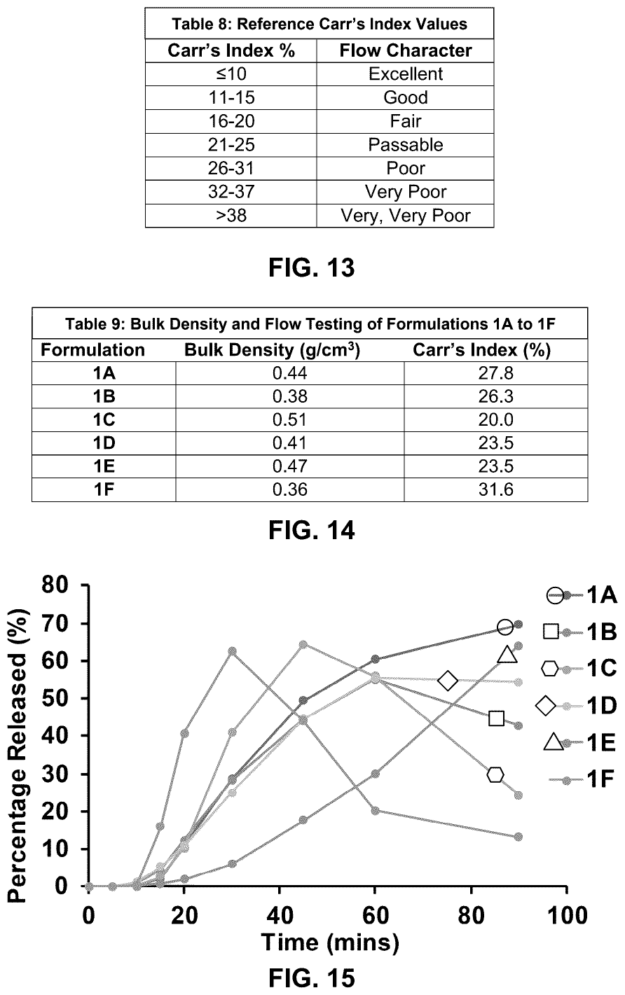

FIG. 13 gives Table 8, showing reference Carr's Index Values.

FIG. 14 is Table 9, the Bulk Density and Flow Testing of Formulations 1A to 1F.

Based on the reference values (Table 8), Formulations 1C, 1D and 1E were considered fair to passable with Formulations 1A and 1B considered poor and Formulation 1F considered very poor (Table 9). It should be borne in mind that the small-scale manufacture is not entirely representative of flow properties of the blends at larger scales and, therefore, no conclusions with respect to the selection of excipients is supported from only these data. However, the data did suggest that manufacture of NTP42:KVA4 granules suitable for roller compaction was feasible.

The prototype formulations were hand-filled into capsules and evaluated for their dissolution profiles in the physiologically relevant media, namely fasted state simulated intestinal fluid (FaSSIF, pH 6.5).

FIG. 15 illustrates dissolution of NTP42 from Prototype Capsules, Formulations 1A to 1F. All Formulations 1A-1F dispersed on capsule breach around 10-15 min of capsule breach with mannitol-based formulations showing faster initial dissolution profiles. While formulations containing sodium starch glycolate (SSG) or sodium lauryl sulfate (SLS) initially released NTP42, precipitation was observed indicating that these excipients are not ideal for DiC NTP42:KVA4.

During the second round of formulation screening, a mannitol-based filler and a crospovidone disintegrant was favored.

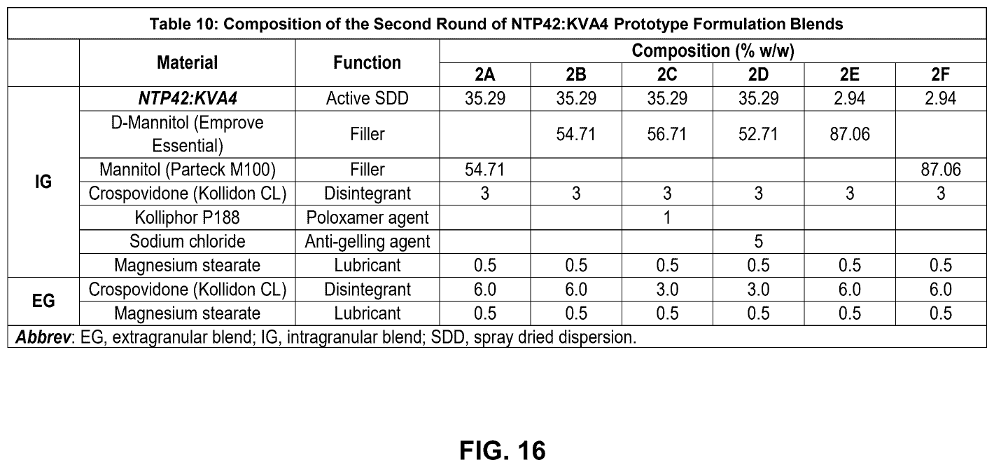

FIG. 16 gives Table 10, Composition of the Second Round of NTP42:KVA4 Prototype Formulation Blends. For comparison, a direct compression grade of mannitol (Partek® M100) was included with the aim of mitigating the risk of poor granule/flow at low SDD loading. Testing also included low dose formulations (formulations 2E and 2F) to cover any intermediate dose strengths. Also, as an alternate to SLS, the poloxamer Kolliphor® P188 was included to assess effect of another wetting agent and the sodium chloride (anti-gelling agent) reduced to 5% w/w. In addition, the extragranular disintegrant was increased to improve initial disintegration on capsule burst.

FIG. 16 gives Table 10, showing results from testing that included: low doses (10 mg NTP42:KVA4) formations (which were 2E & 2F) and Kolliphor® P188 (2C). The formulation blends were assessed for bulk density and flow properties (Carr's Index).

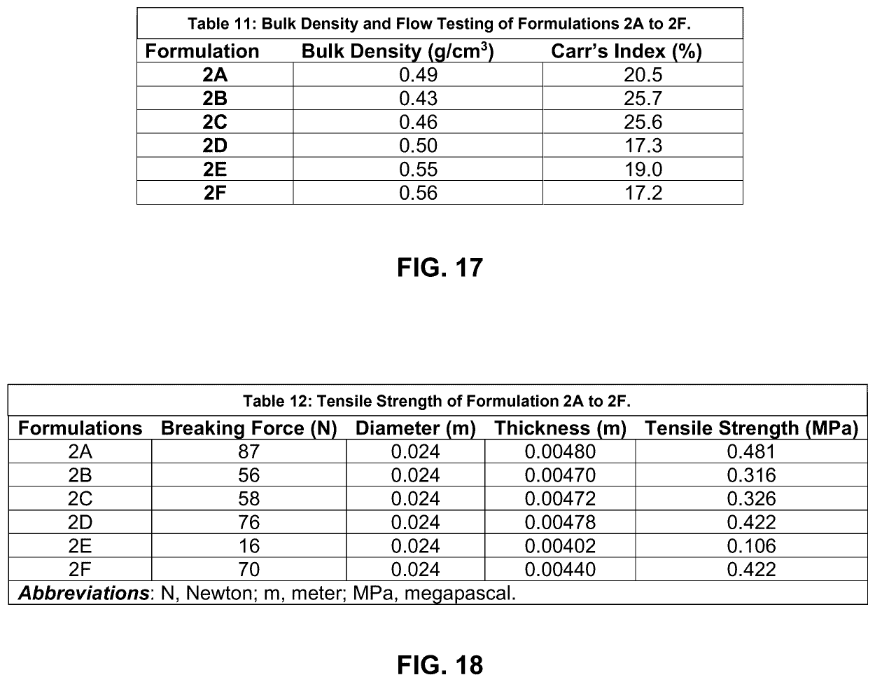

FIG. 17 gives Table 11, showing bulk density and flow properties (Carr's Index) of 2A, 2B, 2C, 2D, 2E, and 2F. Compared to the first round, the second-round formulations had better flow characteristics. According to the Carr's index reference values (Table 8), formulations containing the M100 grade of mannitol (Formulations 2A & 2F) had better flow properties than the Emprove grade (Formulations 2B & 2E). While Formulation 2C containing Kolliphor® P188 had poor flow, it may be attributed to the specific grade of mannitol used rather than the Kolliphor® P188 itself, and it may be permissible to include some amount of, e.g., Kolliphor® P188 or other excipient. Compositions of the disclosure may be described as consisting essentially of: a capsule; 316.20 mg of an ASD+a filler, wherein the 316.20 mg ASD+filler comprises an amount of ASD (e.g., 10.00 mg, 29.99 mg, 119.99 mg), with a balance of the 316.20 mg being the filler, wherein the filler is mannitol; 20.40 mg of crospovidone as the disintegrant; 3.40 mg of magnesium stearate as the lubricant. The ASD is substantially about 1:4 (w/w) active ingredient:copovidone, wherein the active ingredient is one of the substituted biphenyl benzenesulfonyl urea compounds shown herein such as TP20, NTP42, NTP43, or NTP48, most preferably NTP42. This description includes the proviso that the described composition contains no microcrystalline cellulose (MCC) (but could contain some minor amount of other excipient, such as <about 2% of Kolliphor® P188).

To address any potential for issues to arise during roller compaction for formulations with low drug loadings due to a lack of a plastically deforming component in the formulation, Formulations 2A to 2F were also tested for tensile strength of the compacted ribbons using the Dr Schleuniger hardness test.

FIG. 18 gives Table 12, showing data for the Tensile Strength of Formulation 2A to 2F from the hardness test. The low dose formulation containing the M100 grade of mannitol, Formulation 2F, had tensile strength comparable to the high dose formulations, i.e., Formulations 2A-2D. On the other hand, Formulation 2E, the low dose formulation containing a different grade of mannitol, had poor tensile strength and visually the slugs were brittle. M100 is an art-recognized direct compaction grade of spray-dried mannitol and generally an example of a “direct compaction grade mannitol” discussed in Kosugi, 2020, Effect of different direct compaction grades of mannitol on the storage stability of tablet properties investigated using a Kohonen self-organizing map and elastic net regression model, Pharmaceutics 12(9):886, incorporated by reference. Thus, for the low dose at least the M100 grade of mannitol (e.g., “direct compaction grade mannitol”) is preferred.

To address any potential risk that low dose blends may not be uniform with respect to the drug or excipient content, Formulations 2E and 2F were also evaluated for blend uniformity by tray sampling. In brief, the entire formulation was spread out on a tray and sampled at nine pre-defined locations on the tray. Each of the nine samples were analyzed to confirm that they met the expected or target assay levels of NTP42 for a capsule with a dose strength of 10 mg NTP42:KVA4 (2 mg NTP42). For both Formulations 2E & 2F, all nine measurements met the predefined acceptance criteria with the mean blend uniformity ≤5.0% relative standard deviation (RSD).

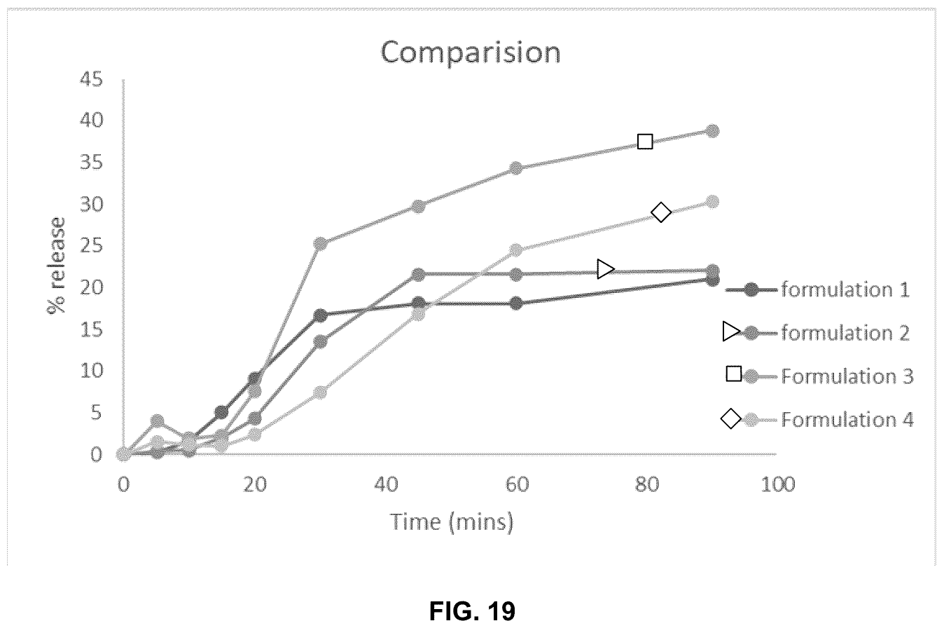

The high dose formulations, Formulations 2A to 2D (depicted in FIG. 19 as Formulations 1 to 4, respectively), were assessed for their dissolution in FaSSIF pH 6.5 media.

FIG. 19 shows dissolution Profiles of Formulations 2A to 2D in FaSSIF, pH 6.5.

Based on the bulk density, tensile strength and the dissolution profiles of the Formulations 2A-2E in FaSSIF along with blend uniformity of the low dose (Formulations 2E & 2F), two formulations were selected for capsule development. For the optimized prototypes, low and high doses were evaluated, i.e., 10 mg and 120 mg NTP42:KVA4 per capsule.

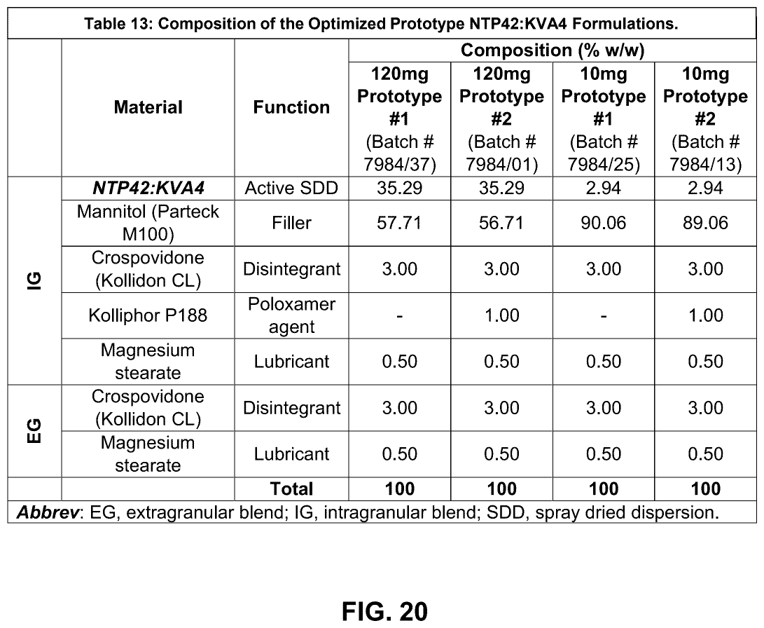

FIG. 20 gives Table 13, showing compositions of the optimized prototypes of 10 mg and 120 mg NTP42:KVA4 per capsule that were manufactured. For the manufacturing process the components were layered into the blending vessel as follows:
⅓ M100 Mannitol.
Half of NTP42:KVA4.
Half of Kollidon® CL
⅓ M100 Mannitol.
Half of NTP42:KVA4.
Half of Kollidon® CL.
⅓ M100 Mannitol

Materials were blended for 15 mins at 30 rpm and the resulting mixture passed through a sieve before being tested for blend uniformity. The extragranular excipients Kollidon® CL and magnesium stearate were then added and the blends were subject to roller compaction trials.

The low (10 mg NTP42:KVA4, Batch 7984/25) and high (120 mg NTP42:KVA4, Batch #7984/37) dose strengths of Prototype #1 were assessed in roller compaction trials to optimize screw feeder and roll pressure settings for optimum ribbon and granule properties. Both dose strengths produced granules that were suitable for scale-up with favorable density and flow properties. Blends were filled into capsules and assessed for dissolution in FaSSIF as well as in phosphate buffer, pH 6.8. In both the FaSSIF media and phosphate buffer, NTP42 was efficiently released from the capsules of the low and high dose strengths.

At 250 g scale, optimized prototypes (Table 13) were manufactured, and capsules filled using an automated capsule filler. The resulting capsules for each prototype and at each dose strength were then packed into 60 mL high density polyethylene (HDPE) bottles (e.g., DUMA®) with 4 g desiccant (integral part of lid), child-resistant closures and set down for two months stability at refrigerated (2-8° C.), 25° C./60% RH and at 40° C./75% RH.

Based on initial (T=0M) and one month stability data (T=1M), Prototype #1 from Table 13 was selected as the lead to be brought into a manufacturing. Thus is provided DiC formulations of substituted biphenyl benzenesulfonyl urea thromboxane receptor antagonists compounds (such as N-(tert-butylcarbamoyl)-5-cyano-2-((4′-(trifluoromethoxy)-[1,1′-biphenyl]-3-yl)oxy)benzenesulfonamide) as an active ingredient in an amorphous solid dispersion (ASD) with copovidone, in which the ASD with a disintegrant and lubricant make up an intragranular blend that, along with an extragranular disintegrant/lubricant blend are encapsulated, making a drug-in-capsule (DiC) formulation.

1. A pharmaceutical composition comprising:
a formulation blend comprising (i) an amorphous solid dispersion (ASD) comprising an active ingredient and copovidone, (ii) a filler, (iii) a disintegrant, and (iv) a lubricant, wherein the composition is contained in a capsule and wherein the active ingredient comprises a compound of formula (I)

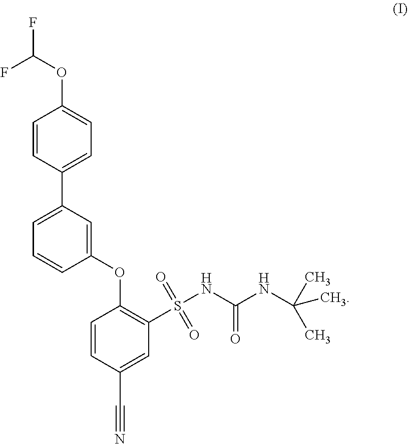 [SMILES: CC(C)(C)NC(=O)NS(=O)(=O)c1cc(C#N)ccc1Oc1cccc(-c2ccc(OC(F)F)cc2)c1]

2. The composition of claim 1, wherein the active ingredient and the copovidone are present in the ASD at a ratio of 1:4 w/w.

3. The composition of claim 1, wherein the formulation blend consists essentially of (i) the amorphous solid dispersion (ASD) consisting essentially of the active ingredient and the copovidone, (ii) the filler, (iii) the disintegrant, and (iv) the lubricant.

4. The composition of claim 1, further comprising a poloxamer agent.

5. The composition of claim 1, wherein the formulation blend comprises:
93.0% the ASD and the filler;
6.0% of the disintegrant; and
1.0% of the lubricant.

6. The composition of claim 4, wherein the formulation blend consists essentially of:
92.0% the ASD and the filler;
the 1.0% poloxamer agent;
6.0% of the disintegrant; and
1.0% of the lubricant.

7. The composition of claim 1, wherein the formulation blend comprises:
an intragranular blend comprising (i) the amorphous solid dispersion (ASD) comprising the active ingredient and the copovidone, (ii) the filler, (iii) a first quantity of the disintegrant, and (iv) a first quantity of the lubricant; and
an extragranular blend comprising the intragranular blend with (v) a second quantity of the disintegrant, and (vi) a second quantity of the lubricant.

8. The composition of claim 1, wherein the composition comprises:
an intragranular blend comprising (i) the amorphous solid dispersion (ASD) comprising the active ingredient and copovidone, (ii) the filler, (iii) a poloxamer agent, (iv) a first quantity of the disintegrant, and (v) a first quantity of the lubricant; and
an extragranular blend comprising the intragranular blend with (vi) a second quantity of the disintegrant, and (vii) a second quantity of the lubricant.

9. The composition of claim 7, wherein the composition comprises:
93.0% the ASD and the filler;
3.0% the first quantity of the disintegrant;
0.5% the first quantity of the lubricant;
3.0% the second quantity of the disintegrant; and
0.5% the second quantity of the lubricant.

10. The composition of claim 8, wherein the composition comprises:
92.0% the ASD and the filler;
1.0% the poloxamer agent;
3.0% the first quantity of the disintegrant;
0.5% the first quantity of the lubricant;
3.0% the second quantity of the disintegrant; and
0.5% the second quantity of the lubricant.

11. The composition of claim 5, wherein the ASD is between about 0.1 and about 90% w/w of the composition with the filler providing the balance of the 93.0%.

12. The composition of claim 6, wherein the ASD is between about 0.1 and about 90% w/w of the composition with the filler providing the balance of the 92.0%.

13. The composition of claim 1, wherein the filler is mannitol, the disintegrant is crospovidone, and the lubricant is magnesium stearate.

14. The composition of claim 1, wherein the filler is mannitol, the poloxamer agent is Kolliphor® P188, the disintegrant is crospovidone, and the lubricant is magnesium stearate.

15. The composition of claim 1, wherein the active ingredient is amorphous.

16. A method of formulating an active ingredient, the method comprising:
making an amorphous solid dispersion (ASD) comprising the active ingredient and copovidone;
forming a blend comprising the ASD, a filler, a first quantity of a disintegrant, and a first quantity of a lubricant;
granulating the blend to form a granulated intragranular blend;
preparing a mix comprising the intragranular blend and an extragranular blend that comprises a second quantity of the disintegrant and a second quantity of the lubricant; and
encapsulating the mix in capsules, wherein the active ingredient comprises a compound of formula (I)

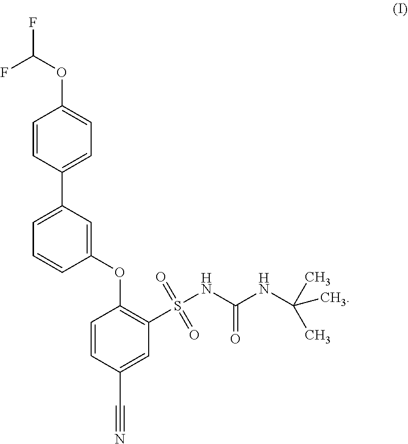 [SMILES: CC(C)(C)NC(=O)NS(=O)(=O)c1cc(C#N)ccc1Oc1cccc(-c2ccc(OC(F)F)cc2)c1]

17. The method of claim 16, wherein the active ingredient and the copovidone are present in the ASD at a ratio of 1:4 w/w.

18. The method of claim 17, wherein the mix consists essentially of:
the ASD at between about 0.1 and about 90% w/w of the mix;
the filler in an amount so that the ASD and the filler are 93.0% w/w of the mix;
3.0% the first quantity of the disintegrant;
0.5% the first quantity of the lubricant;
3.0% the second quantity of the disintegrant; and
0.5% the second quantity of the lubricant.

19. The method of claim 18, wherein the filler is mannitol, the disintegrant is crospovidone, and the lubricant is magnesium stearate.

20. The method of claim 17, wherein the ASD is made by making a spray-dried dispersion.

21. The method of claim 16, wherein the granulating step is performed with a roller compactor.

22. The method of claim 17, wherein an entirety of the active ingredient is amorphous.
